(19) United States
(12) Patent Application Publication
(10) Pub. No.: US 2009/0022825 A1
(43) Pub. Date: Jan. 22, 2009

(54) Method for Predicting Whether Subjects With Mild Cognitive Impairment (MCI) Will Develop Alzheimer's Disease

(75) Inventors: Daniel Kerkman, Lake Villa, IL (US); John F. DeBernardis, Lindenhurst, IL (US); Raymond Zinkowski, Northbrook, IL (US); Harald Juergen Hampel, Munich (DE)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: Applied NeuroSoloutions

(21) Appl. No.: 12/114,087

(22) Filed: May 2, 2008

Related U.S. Application Data
(63) Continuation of application No. 10/704,233, filed on Nov. 7, 2003, now abandoned.
(60) Provisional application No. 60/424,628, filed on Nov. 7, 2002.

(51) Int. Cl. A61K 36/16; C12Q 1/00; A61P 25/28

(52) U.S. Cl. 424/752; 435/4

(57) ABSTRACT

The invention provides methods of diagnosing mild cognitive impairment (MCI) in a patient and methods of predicting cognitive decline in patients who have MCI. The invention also provides methods for predicting if a patient who has MCI will develop Alzheimer's Disease (AD).

This application is related to and claims the benefit of U.S. Provisional Application Ser. No. 60/424,628 filed Nov. 7, 2002, the disclosure of which is incorporated by reference herein.

### FIELD OF THE INVENTION

The invention relates to methods of diagnosing mild cognitive impairment (MCI) in a patient and predicting the rate of cognitive decline in patients who have MCI. Specifically, the invention relates to methods of predicting if a patient who has MCI will develop Alzheimer's Disease (AD). More specifically, the invention relates to determining if a patient with MCI is likely to develop AD comprising measuring the level of tau phosphorylated at Threonine 231 (p-tau₂₃₁) in the cerebrospinal fluid (CSF) of the patient.

### BACKGROUND OF THE INVENTION

Cross-sectional Alzheimer's Disease (AD) studies typically demonstrate elevated Cerebrospinal fluid (CSF) levels of the microtubule associated protein, tau (Vanmechelen et al., 2001, Mech. Aging Dev. 122:2005-2011). Surprisingly, in spite of the well characterized progressive clinical decline and the increasing topography of brain involvement, equal numbers of studies show that the CSF tau levels in AD are progressive (Arai et al., 1997, JAGS 45:1228-1231; Blomberg et al., 1996, Neurosci. Lett. 214:163-166; Hampel et al., 2001, Ann. Neurol. 49:545-546; Kanai et al., 1999, Neurosci. Lett. 267:65-68) as are not (Andreasen et al., 1999, Neurology 53:1488-1494; Nishimura et al., 1998, Methods Find. Exp. Clin. Pharmacol. 20:227-235; Sunderland et al., 1999, Biol. Psychiatry 46:750-755; Tapiola et al., 2000, Neurosci. Lett. 280:119-122). Thus, whether the CSF tau level reflects neuronal damage is unclear from these studies.

Phosphorylation at threonine 231 of tau has been shown to be specific for AD and to precede assembly of paired helical filaments in human brain biopsy tissue (Vincent et al., 1998, Neurobio. Aging 19:87-97). AD is the leading cause of dementia, and patients with AD have elevated CSF p-tau₂₃₁ levels compared with control subjects (Kohnken et al., 2000, Neurosci. Lett. 287:187-190).

Mild Cognitive Impairment (MCI) is a condition characterized by mild recent memory loss without dementia or significant impairment of other cognitive functions that are beyond what is normal for age or educational background. Many patients with MCI progress to AD. Two recent mild cognitive impairment (MCI) studies using the diagnostically more specific measure of hyperphosphorylated tau (p-tau), reported cross-sectional elevations, but did not observe longitudinal change (Arai et al., 2000, Exp. Neurol. 166:201-203; Buerger et al., 2002, Neurology 59: 627-629). Since MCI is a major risk factor for Alzheimer's Disease (AD) (Petersen et al., 1995, JAMA 273:1274-1278), predicting cognitive decline in patients with MCI is highly desirable to effectively determine which patients might benefit from disease-modifying, anti-dementia drugs. The invention provides methods for predicting which patients are likely to develop AD by measuring the levels of p-tau₂₃₁ in the CSF.

### SUMMARY OF THE INVENTION

The invention provides methods of diagnosing mild cognitive impairment (MCI) in a patient and methods of identifying patients who are likely to cognitively deteriorate and develop dementia. Specifically, the invention provides a method of predicting whether a patient will develop Alzheimer's Disease (AD), particularly a patient who has MCI. The invention also provides a method for determining the stage of neuronal degeneration in a patient who has AD comprising measuring the level of p-tau₂₃₁ in the CSF of the patient with AD.

In certain aspects, the method of the invention comprises taking a sample of cerebrospinal fluid (CSF) from a patient and measuring the level of phosphorylated tau (p-tau) protein in the CSF, wherein a level of p-tau that is at least about 215 pg/ml is indicative of a patient who will develop or has AD. Preferably, the level is at least about 250 pg/ml. More preferably, the level is at least about 300 pg/ml. Most preferably, the level is at least about 350 pg/ml.

In another aspect, the method further comprises determining the stage of cognitive deterioration of the patient by comparing the level of CSF p-tau with the level of CSF p-tau obtained from at least one control patient, wherein the greater the difference between the level of CSF p-tau from the patient and the level of CSF p-tau from the control patient, the greater the risk of developing AD.

In some aspects, the p-tau can be a tau protein that is phosphorylated at any of amino acids 175, 181, 185, 199, 202, 214, 231, 235, 262, 396, 404, 409, and 422. Preferably, the methods of the invention measure the level of tau phosphorylated at threonine 231 (p-tau₂₃₁), 181, 396, or 404. Most preferably, the methods of the invention measure the level of p-tau₂₃₁.

The invention also provides a method of predicting the rate of neuronal degeneration in a patient comprising the step of determining the level of tau protein phosphorylated at threonine 231 (p-tau₂₃₁) in a sample of cerebrospinal fluid (CSF) taken from the patient, wherein the patient has Alzheimer's Disease.

The invention also provides methods of identifying a patient who is likely to develop Alzheimer's Disease comprising: (a) obtaining a biological sample from a patient; (b) determining the level of phosphorylated tau protein (p-tau) in the biological sample; (c) identifying the patient as being likely to develop Alzheimer's Disease if the level of p-tau in the biological sample is about 617 pg/ml or higher.

In addition, the invention provides methods of predicting cognitive decline in a patient, comprising the steps of: (a) obtaining a biological sample from a patient; (b) determining the level of phosphorylated tau protein (p-tau) in the biological sample; (c) identifying the patient as being likely to cognitively decline if the level of p-tau in the biological sample is about 143 pg/ml or higher.

Further, the invention provides methods of diagnosing Alzheimer's Disease in a patient, comprising the steps of: (a) obtaining a biological sample from a patient; (b) determining the level of p-tau protein in the biological sample; and (c) diagnosing the patient as having Alzheimer's Disease if the level of p-tau in the biological sample is about 215 pg/ml or higher.

The invention also provides methods of monitoring cognitive decline in a patient comprising the steps of: (a) determining the level of p-tau protein in a biological sample obtained from the patient; (b) determining the p-tau load in the sample by adjusting the p-tau level using a ventricular volume correction; (c) repeating steps (a) and (b) using a subsequently-collected biological sample obtained from the patient; and (d) comparing the p-tau load determined in step (b) with the amount of the p-tau load determined in step (c) and therefrom monitoring cognitive decline in the patient.

The invention also provides methods of measuring the effectiveness of a pharmaceutical composition as an agent for treating a patient having a condition associated with dementia, comprising the steps of: (a) determining the level of p-tau protein in a biological sample obtained from the a patient; (b) administering an amount of a pharmaceutical composition to the patient; (c) repeating step (a) using a subsequently-collected biological sample obtained from the patient; (d) comparing the level of p-tau protein determined in step (a) with the level of p-tau protein determined in step (c), wherein the effectiveness of the pharmaceutical composition is monitored by detecting no change in the level or p-tau or a decrease in the level of p-tau in the subsequently-collected biological sample compared with the biological sample from step (a).

In certain aspects, the methods of the invention can be used to identify patients in need of Alzheimer's therapy. For example, once a patient is identified, by a method of the invention, as having or likely to develop Alzheimer's Disease, or is identified as having MCI, or is identified as being likely to cognitively decline, that patient can be subjected to a therapeutic regime for treating Alzheimer's or other dementias. An Alzheimer's therapy can include, for example, administering an anti-dementia drug to the patient. A non-limiting example of an anti-dementia drug is an anticholinesterase inhibitor (also referred to herein as an anticholinesterase drug).

Also, the invention provides methods of predicting a rate of neuronal degeneration in a patient comprising determining the level of phosphorylated tau (p-tau) protein in a sample of cerebrospinal fluid (CSF) taken from the patient, wherein the patient has Alzheimer's Disease.

In certain aspects, p-tau, as used herein, refers to tau protein phosphorylated at amino acid 175, 181, 185, 199, 202, 214, 231, 235, 262, 396, 404, 409, or 422.

In other aspects, a biological sample used in methods of the invention is cerebrospinal fluid (CSF).

Specific preferred embodiments of the invention will become evident from the following more detailed description of certain preferred embodiments and the claims.

### DETAILED DESCRIPTION OF THE DRAWINGS

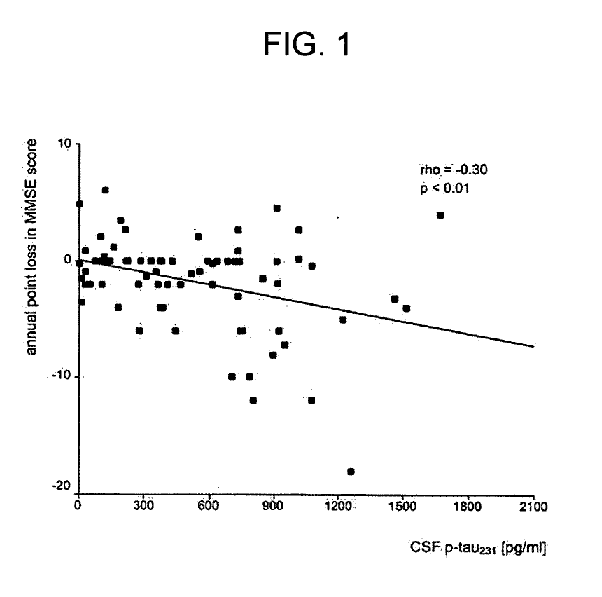

FIG. 1 depicts a graph showing the correlation between level of p-tau₂₃₁ in CSF at baseline and annual point loss in MMSE score for 77 patients with mild cognitive impairment (MCI) who were observed during a longitudinal study.

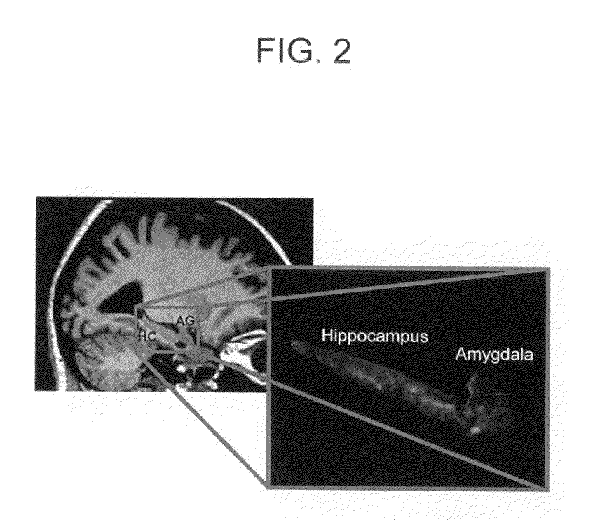

FIG. 2, left panel, shows the right hippocampus (hc) and amygdala (ag) manually outlined in a sagittally oriented volumetric MRI section illustrating the localization of both structures. FIG. 2, right panel, shows a view from right anterior-superior on the right hippocampus and amygdala after manual editing. The depicted MRI scan stems from an AD patient. Accordingly the head of the hippocampus and the amygdala show severe atrophy, particularly on the right hand side of the figure.

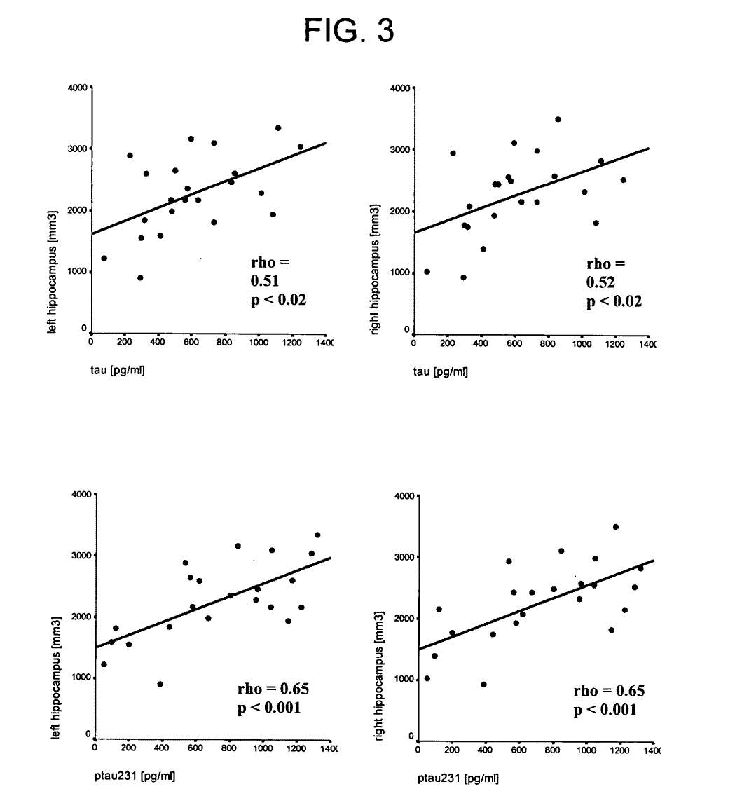

FIG. 3 depicts a graph showing that higher levels of CSF tau proteins [pg/ml] correlate with higher baseline volumes of right and left hippocampus [mm³] in AD patients.

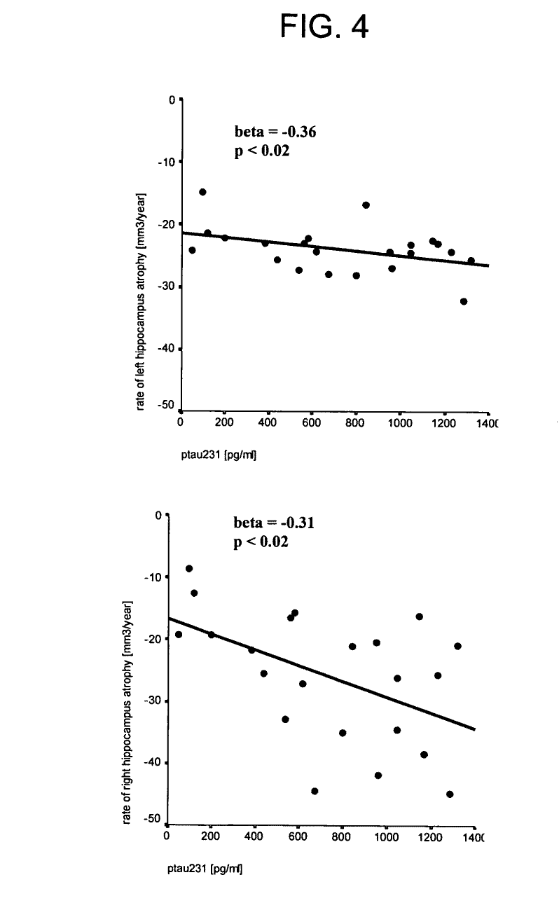

FIG. 4 depicts a graph showing that higher CSF p-tau₂₃₁ levels at baseline [pg/ml] correlate with higher annual rates of right hippocampus atrophy [mm³/year] derived from a mixed effects regression model in AD patients.

### DETAILED DESCRIPTION OF THE PREFERRED EMBODIMENTS

Kohnken et al. developed an immunoassay designed to specifically detect tau protein phosphorylated at threonine 231 (p-tau₂₃₁) that can discriminate between patients with AD and control subjects with high accuracy (see Kohnken et al., 2000, Neurosci. Lett. 287:187-190). Using the immunoassay, the group also demonstrated that concentrations of p-tau in the cerebrospinal fluid (CSF) correlate with disease progression in individual patients (Hampel et al., 2001, Ann. Neurol. 49:545-546), suggesting that p-tau₂₃₁ is a potential biomarker of AD progression. Neuropathologic evidence demonstrated that phosphorylation of tau at threonine 231 occurs very early in AD pathophysiology (Vincent et al., 1998, Neurobiol. Aging 19:287-296). As described herein, p-tau₂₃₁ concentrations can also be used to identify individuals who are likely to develop Alzheimer's Disease, particularly individuals who have been previously diagnosed as having mild cognitive impairment (MCI).

As demonstrated in the Examples, p-tau₂₃₁ levels are elevated in the cerebrospinal fluid (CSF) of patients who have mild cognitive impairment (MCI) compared with healthy control patients (HC). High p-tau₂₃₁ levels at baseline (i.e. p-tau₂₃₁ levels at the beginning of the study) correlated with the annual point loss in Mini-Mental State Examination (MMSE) scores in patients with MCI, while no correlation was observed with annual point loss in MMSE and total tau (t-tau) levels, which is a nonspecific marker of neurodegeneration (Blennow et al., 2001, Mol. Neurobiol 24:87-97). A subgroup of the MCI patients in the studies described in the Examples converted to Alzheimer's Disease (AD). Consistent with the analysis of rates of cognitive decline, increased levels of p-tau₂₃₁ correlated with conversion to AD, while t-tau levels did not.

The literature shows considerable difficulty in detecting tau concentration changes during the course of AD and in MCI (Arai et al., 2000, Exp. Neurol. 166:201-203; Buerger et al., 2002, Neurology 59: 627-629). In a review of CSF proteins, Reiber observed a higher ventricular to lumbar level ratio for brain-derived proteins (e.g. tau 1.5:1 and S-100B 18:1) with lower ratios for systemic derived proteins (e.g. albumin 1:205) (Reiber, 2001, Clin. Chim. Acta. 310:173-186). Amyloid beta (Aβ) levels are higher in lumbar than ventricular CSF (1:2) possibly reflecting its central and peripheral sources. In one embodiment, the invention provides a method for monitoring CSF p-tau levels longitudinally (i.e. over a period of time) comprising the step of determining the level of phosphorylated tau protein (p-tau) at various time points and adjusting the level of p-tau at each time point based on the ventricular volume. The level adjustment is referred to herein as a “ventricular volume correction.”

The ventricular volume correction is accomplished by measuring the level of p-tau in the CSF of a patient, multiplying the p-tau concentration (pg/ml) by the ventricular volume (ml) and dividing by 1000. Ventricular volume can be determined, for example, by acquiring magnetic resonance imaging (MRI) scans from a patient and converting the MRI signal intensities to ventricular volume using an automated protocol as described by Fox and Freeborough (1997, J. Mag. Reson. Imag. 7:1069-1075). An adjusted p-tau level is referred to herein as a p-tau load. As demonstrated in Example 3 below, when the adjustment for ventrticular volume was made, the p-tau₂₃₁ load showed a significant longitudinal group-by-time interaction. The follow-up tests showed that the p-tau₂₃₁ load increased in patients with MCI, but did not change in the control group. Moreover, regression analyses of the longitudinal data showed that measuring p-tau₂₃₁ load changes significantly improved the prediction of group membership over measuring p-tau₂₃₁ level changes.

The rationale for using the ventricular volume correction is based on the understanding that tau levels are higher in the ventricular CSF than in the lumbar CSF. As demonstrated herein, ventricular volume correction is useful only for the p-tau₂₃₁ measurement. Thus, even though both Aβ40 and p-tau₂₃₁ levels are elevated in MCI, after applying the ventricular volume correction, a longitudinal change (i.e. change over time) is observed only for the p-tau load in the MCI group.

In one embodiment, the invention provides a method that comprises using the ventricular volume correction to monitor cognitive decline in a patient. The method comprises the steps of determining the p-tau load, as described above and in the Examples, in a biological sample (preferably CSF) from the patient at various time points. For example, samples can be obtained upon diagnosis of a neurological disorder, such as mild cognitive impairment (MCI) or Alzheimer's Disease (AD), and at various times subsequent to diagnosis, such as every 6 months or annually. Monitoring cognitive decline allows a care provider to determine if a patient is responding well to certain treatments.

In another embodiment, the level of p-tau can be measured in a patient at various times during treatment for MCI or a condition associated with dementia. For example, levels of p-tau, preferably p-tau₂₃₁, can be measured at the beginning of treatment with anti-dementia drugs, such as anticholinesterase drugs. Non-limiting examples of anticholinesterase drugs include tacrine, donepezil, rivastigmine, and galantamine. The level of p-tau can then be monitored at various times, such as weekly, monthly, bi-monthly, or any other chosen time periods, during treatment to determine if the drugs are effective. An effective drug will cause the level of p-tau to decrease over time.

Alternatively, an effective drug will be identified if the level of p-tau does not increase over time. Non-limiting examples of conditions associated with dementia include, but are not limited to, Alzheimer's Disease (AD), frontotemporal dementia (FTD), vascular dementia, Lewy body dementia, or other neuorological disorders.

In one embodiment, the invention provides a method for identifying a patient who has or is likely to develop AD. In a particular embodiment, the method comprises the steps of taking a biological sample (preferably a sample of CSF) from a patient, determining the p-tau level (preferably p-tau₂₃₁) in the biological sample, and diagnosing the patient with AD if the p-tau level is about 215 pg/ml or higher. The patient can be an individual who has been diagnosed as having mild cognitive impairment (MCI) or any form of dementia, such as frontotemporal dementia (FTD), vascular dementia, Lewy body dementia, or other neuorological disorders. Alternatively, the patient can be an individual who desires or requires screening for AD but who has not been diagnosed with any neurological disorders, dementias, or MCI.

In another embodiment, a method of the invention comprises the steps of taking a biological sample (preferably a sample of CSF) from a patient, determining the p-tau level (preferably p-tau₂₃₁) in the biological sample, and identifying the patient as being likely to develop AD if the level of p-tau in the sample is about 617 pg/ml or higher. In this embodiment, the level of p-tau indicates a patient who is likely to convert to AD from a more mild neurological disorder. For example, the patient can be diagnosed as having MCI prior to being subjected to a method of the invention.

In yet another embodiment, the invention provides a method of predicting cognitive decline in a patient comprising the steps of taking a biological sample (preferably a sample of CSF) from a patient, determining the p-tau level (preferably p-tau₂₃₁) in the biological sample, and identifying the patient as being likely to develop AD if the level of p-tau in the sample is about 143 pg/ml or higher. Preferably the patient has been diagnosed as having MCI.

In another embodiment, a method of the invention comprises the steps of taking a biological sample (preferably a sample of CSF) from a patient, determining the p-tau₂₃₁ level in the biological sample, and associating the p-tau₂₃₁ level with a particular stage of cognitive decline. In another embodiment, the p-tau₂₃₁ level can be adjusted to determine the p-tau₂₃₁ ventricular load by correcting the level for CSF ventricular volume, and the p-tau₂₃₁ load can be associated with a particular stage of cognitive decline.

Any biological sample, such as tissues, cells, or body fluids, can be used in a method of the invention, so long as the sample comprises phosphorylated tau (p-tau) protein. In a preferred embodiment, the biological sample used in any method of the invention is cerebrospinal fluid (CSF). Samples of CSF can be collected by any means commonly used by those skilled in the art. For example, CSF can be collected by lumbar puncture using fluoroscopy to guide a beveled LP needle into the subarachnoid space.

The level of phosphorylated tau proteins (p-tau) can be determined in a method of the invention using, for example, an immunoassay or protein-binding assay. Preferably, p-tau₂₃₁ levels are measured as described herein, using a sandwiched enzyme-linked immunosorbent assay (ELISA) to capture tau protein, then detecting phosphorylation of tau with a phosphorylated tau specific antibody (e.g. CP9, which is an antibody specific for p-tau₂₃₁) (see Kohnken et al., 2000, Neurosci. Lett. 287:187-190).

A ventricular volume correction can be accomplished as described above to determine the p-tau₂₃₁ load. The ventricular volume can be determined, for example, as described in Example 3 below.

In one embodiment, a method of the invention can be used to determine if a patient has MCI or AD, based on the ventricular p-tau₂₃₁ load. In some cases, the patient can be previously diagnosed as having MCI, and the method of the invention can be used to determine the likelihood of the condition to progress to AD. In another embodiment, a patient can be examined at follow-up intervals to assess the progression of MCI.

The methods of the invention can be used alone or in combination with any known diagnostic test(s) and/or marker(s) for diagnosing AD or MCI or for identifying or monitoring cognitive decline, including, but not limited to, mini mental status examination (MMSE), global deterioration scale (GDS) scores, age, amyloid beta, psycohometric test battery (Kluger et al. 1999, J. Geriat. Psychiat. Neurol. 12:168-179), the Guild Memory Test, the Wechsler adult intelligence scale test, magnetic resonance imaging (MRI), and APOE genotype. Combination with methods of the invention can increase the sensitivity and decrease the number of false positives of known tests and markers for AD, MCI, and cognitive decline.

In one embodiment, the invention provides methods for examining neuronal degeneration in patients having Alzheimer's disease (AD). AD is characterized by progressive loss of specific neuron populations. The temporal sequence of regional neuron loss is not yet well established. Neuropathological and structural imaging evidence, however, suggests that allocortical areas, including entorhinal cortex, hippocampus and amygdala, are among the earliest affected brain regions in AD.

In clinical-pathological studies, measurement of hippocampal volume based on magnetic resonance imaging (MRI) accounts for about 80% of variability in hippocampal neuron density in AD. These findings support the notion that in vivo measurement of hippocampus volume can serve as an indirect measure for the extent of allocortical neuronal degeneration in AD. Magnetic resonance imaging (MRI) studies showed significant atrophy of the hippocampus even in preclinical stages of AD. Hippocampus atrophy is the most extensively investigated marker of intra-individual structural disease progression in AD in longitudinal MRI studies.

In one embodiment, the invention provides an in vivo surrogate measure of AD-related neuronal destruction comprising measuring the level of p-tau₂₃₁ in CSF of patients who have AD. As described in Example 4 below, baseline hippocampus volumes were significantly reduced in AD patients compared to controls (p<0.005). CSF p-tau₂₃₁, but not t-tau, concentrations at baseline correlated with rates of atrophy of left and right hippocampus (p<0.02). In addition, there was a significant positive correlation between tau-proteins and baseline hippocampus volumes (p<0.03). The rates of cognitive decline correlated significantly with rates of hippocampus atrophy (p<0.05).

The following Examples are provided for the purposes of illustration and are not intended to limit the scope of the present invention. The present invention is not to be limited in scope by the exemplified embodiments, which are intended as illustrations of individual aspects of the invention. Indeed, various modifications of the invention in addition to those shown and described herein will become apparent to those skilled in the art from the foregoing description and accompanying drawings. Such modifications are intended to fall within the scope of the appended claims.

### EXAMPLES

### Example 1

### CSF P-tau₂₃₁ Correlates with Cognitive Decline in Mild Cognitive Impairment (MCI) Subjects

Seventy-seven subjects having symptoms of mild cognitive impairment (MCI) were selected for a longitudinal study to analyze various biomarkers for MCI. Thirty-nine of the MCI subjects cognitively deteriorated during follow-up (MCI decliners; annual point loss in Mini-Mental State Examination (MMSE) score of <0). MMSE was assessed as described in Folstein et al., 1975, J. Psyshiatr. Res. 12:189-198. Twenty-six of the MCI decliners converted to probable Alzheimer's Disease (AD) (NINCDS-ADRDA criteria; McKhann et al., 1984, Neurology 34:939-44). For group comparisons, 55 patients with probable AD and 30 healthy control subjects (HC) were also analyzed. Data for patients with AD and HC were collected at baseline.

Patients fasted overnight, and samples of cerebrospinal fluid (CSF) were collected from each patient using fluoroscopy to guide a 22 gauge beveled LP needle. Phosphorylated tau (p-tau) protein levels were measured by an enzyme-linked immunosorbent assays (ELISA) as described in Kohnken et al. (Kohnken et al., 2000, Neurosci. Lett. 287:187-190) using an anti-p-tau₂₃₁ antibody (Molecular Geriatrics Corporation, Vernon Hills, Ill., USA). Levels of t-tau were measured by ELISA using the Innotest hTau kit (art. no. K-1032; Innogenetics, Zwjindrecht, Belgium). Data were reported as means±SD. The APOE genotype was determined according to standard procedures.

The yearly point loss in MMSE score was modeled with stepwise multiple linear regression to generate a multiple regression model (see FIG. 1). The independent variables were baseline p-tau₂₃₁ levels, MMSE score, age, as well as sex, APOE genotype as a two-level variable (0 vs ½ ε4 alleles), center, treatment with cognitive enhancers, and time of follow-up. In a second multiple linear regression model, p-tau₂₃₁ was replaced by t-tau. To establish cutoff levels for tau proteins to differentiate between MCI decliners and HC, sensitivity and specificity levels were set at >80% as recommended by a consensus report from The Ronald and Nancy Reagan Research Institute on Aging Working Group, (1998, Neurobiol. Aging 19:109-116).

Characteristics of patients and HC are given in Table 1. p-tau₂₃₁ and t-tau levels were higher (p<0.001) in patients with MCI than in HC (see Table 1). For a subgroup matched for age (p=0.27) of 30 patients with MCI (age, 63.6±4.4 years) and 30 HC, differences in baseline levels of p-tau₂₃₁ (age-matched patients with MCI, 430±436 pg/mL; HC, 78±114 pg/mL) and t-tau (age-matched patients with MCI, 508±243 pg/mL; HC, 247±137 pg/mL) remained stable (p<0.001).

TABLE 1

| Group (no. | Mean age | | Mean MMSE | Mean CSF | Mean CSF |
|---|---|---|---|---|---|
| of | in y ± SD* | Females/Males, | score ± SD^{†} | p-tau₂₃₁ level, | t-tau level, |
| subjects) | (range) | n | (range) | pg/mL ± SD^{‡} | pg/mL ± SD§ |
| AD (55) | 71.0 ± 6.7 | 31/24 | 18.0 ± 2.9 | 710 ± 373 | 721 ± 352 |
| | (53-84) | | (13-22) | | |
| MCI (77) | 71.4 ± 7.9 | 38/39 | 28.1 ± 1.8 | 501 ± 400 | 578 ± 258 |
| | (54-87) | | (23-30) | | |
| HC (30) | 61.3 ± 10.2 | 15/15 | 29.3 ± 0.7 | 78 ± 114 | 247 ± 137 |
| | (44-79) | | (27-30) | | |

*Differences in age between groups: AD vs HC, p < 0.001; MCI vs HC, p < 0.001.
^{†}Differences in MMSE score between groups: AD vs HC, p < 0.001; AD vs MCI, p < 0.001; MCI vs HC, p < 0.001.
^{‡}Differences in CSF p-tau₂₃₁ levels between groups: AD vs HC, p < 0.001; AD vs MCI, p < 0.001; MCI vs HC, p < 0.001.
§Differences in CSF t-tau levels between groups: AD vs HC, p < 0.001; AD vs MCI, p = 0.037; MCI vs HC, p < 0.001.

Thirty-nine patients with MCI cognitively deteriorated over time (MCI decliners: p-tau₂₃₁ level, 590±414 pg/mL; t-tau level, 611±260 pg/mL); 38 patients with MCI had no cognitive decline (p-tau₂₃₁ level, 409±368 pg/mL; t-tau level, 544±254 pg/mL). P-tau₂₃₁ levels were higher (p=0.037) in patients with MCI who converted to AD during follow-up (MCI converters; 617±378 pg/mL) than in nonconverters (442±402 pg/mL). No difference in t-tau levels (p=0.20) between MCI converters (615±212 pg/mL) and nonconverters (559±278 pg/mL) was observed.

Tau protein levels were lower in the MCI group than in patients with AD (p-tau₂₃₁ level, p<0.001; t-tau level, p=0.037). No difference was found between patients with AD and MCI decliners for p-tau231 levels (p=0.13) and t-tau levels (p=0.26). The limits of the 95% CI for the group difference ranged from −43 pg/mL to 281 pg/mL for p-tau₂₃₁ levels and from −22 pg/mL to 242 pg/mL for t-tau levels.

For the 77 patients with MCI followed longitudinally, the mean cognitive decline±SD, as measured by point loss in the MMSE score, was −1.7±4.0 points per year.

A significant correlation between CSF p-tau₂₃₁ levels at baseline and the annual point loss in MMSE score in the MCI group was observed, both in the single effect analysis (Spearman ρ=−0.30; p<0.01; FIG. 1) and after controlling for covariates in the multiple regression model (β=−0.23; p=0.049). Exclusion of three outliers with a very high annual point loss in MMSE score did not change statistical results (Spearman ρ=−0.23; p=0.049). Higher CSF t-tau levels at baseline did not correlate with a higher annual point loss in MMSE score (Spearman ρ=−0.19; p=0.10).

In addition, in the multiple regression model, older age at baseline (β=−0.31; p<0.01) and APOE-ε4 carrier status (p=−0.39; p<0.01) were predictors of cognitive decline. MMSE score at baseline, sex, therapy, time of follow-up, or center did not add a significant amount of explanatory power. The model with p-tau231, APOE-ε4 carrier status, and age explained 27% of variance of the annual point loss in the MMSE score. When p-tau₂₃₁ was replaced by t-tau in the multiple regression model, only age at baseline (p<0.01) and APOE-ε4 carrier status (p<0.001) were predictors of cognitive decline.

The Ronald and Nancy Reagan Research Institute on Aging Working Group concluded in a published consensus report (1998, Neurobiol. Aging 19:109-116) that an ideal biomarker for AD should have a sensitivity of >80% for detecting AD and a specificity of >80% for distinguishing other dementias. At these “a priori” chosen sensitivity and specificity levels of >80% (set in accordance with: The Ronald and Nancy Reagan Research Institute on Aging Working Group, 1998, Neurobiol. Aging 19:109-116), p-tau₂₃₁ (t-tau) discriminated between MCI decliners and HC with a sensitivity of 82% (82%) and a specificity of 87% (80%). The cutoff level for p-tau₂₃₁ (t-tau) was 143 pg/mL (376 pg/mL).

The data indicated that CSF p-tau₂₃₁ levels were elevated in patients with MCI compared with HC. High p-tau₂₃₁, but not t-tau, levels at baseline correlated with the annual point loss in MMSE scores in patients with MCI. Cognitive decline was chosen as an outcome variable because the objective was to identify subjects who will cognitively deteriorate and develop dementia. A subgroup of the MCI patients in this study converted to AD. Consistent with the analysis of rates of cognitive decline, increased levels of p-tau₂₃₁ correlated with conversion to AD, but t-tau levels did not. Therefore, elevated levels of CSF p-tau₂₃₁ at baseline predict cognitive decline in patients with MCI. The contribution of p-tau₂₃₁ levels to the risk of point loss in the MMSE score for patients with MCI is independent from other established risk factors of cognitive decline.

### Example 2

### CSF Levels of p-tau₂₃₁ can Distinguish AD from Other Dementias

One-hundred and ninety-two (192) patients with a clinical diagnosis of AD, frontotemporal dementia (FTD), vascular dementia, Lewy body dementia, or other neuorolgical disorder and healthy controls were selected and levels of t-tau and p-tau₂₃₁ in the CSF were determined as described above. The mean CSF levels of p-tau₂₃₁ were significantly elevated in the AD group compared with all other groups. The level of p-tau₂₃₁ discriminated with a sensitivity of 90.2% and a specificity of 80.0% between AD and all non-AD disorders. Moreover, p-tau₂₃₁ levels improved diagnostic accuracy compared with t-tau levels when patients with AD were compared with healthy controls (P=0.03) and demented subjects (P<0.001), particularly those with FTD (P<0.001), but not those with vascular and Lewy body dementias. The cutoff level of p-tau₂₃₁ yielding the best sensitivity and specificity for discriminating patients with AD from those with non-AD disease and healthy controls was about 215 pg/ml. Thus, a p-tau₂₃₁ CSF level of about 215 pg/ml or higher can distinguish AD from non-AD dementias.

### Example 3

### Ventricular Volume of p-tau₂₃₁ Increases in Patients with Mild Cognitive Impairment

Ten normal elderly volunteers and eight patients (seven MCI and one very mild AD) were subjected at baseline and after 1 year to a screening and diagnostic evaluation that included: medical (history, physical, and laboratory), neurological, psychiatric, and neuropsychological examinations, MRI, and lumbar puncture (LP). The annual evaluations were completed within a 2 month window.

Normal elderly subjects, all volunteers, had global deterioration scale (GDS) scores (assessed as described in Reisberg et al., 1982, Am. J. Psychiatry 139:1136-1139) of one or two (differentiated only by subjective reporting of age-related memory change). MCI patients, all GDS=3, showed clinically recognizable memory impairment in the absence of dementia with the cognitive decline corroborated by a close informant. The very mild AD patient had a GDS=4 and mini mental status examination (MMSE)=27, as determined using the methods described in Folstein et al., 1975, J. Psyshiatr. Res. 12:189-198. At baseline all participants had MMSE scores≧27. Individuals with medical conditions affecting brain structure or function (e.g. stroke, diabetes, head trauma, depression) were excluded.

The psychometric test battery (as described in Kluger et al., 1999, J. Geriat. Psychiat. Neurol. 12:168-179) included evaluation of memory (immediate and 10 min delayed recall from the paragraph and the paired associates subtests of the Guild Memory Test) and attention/psychomotor speed (the Digit-Symbol Substitution Test of the Wechsler adult intelligence scale-revised test).

Magnetic resonance imaging (MRI) scans were acquired using a 1.5 T GE Signa imager. A clinical MRI examination covered the entire brain with contiguous 3 mm axial T2-weighted images. The research scan was a 124 sliceT1-weighted Fast-Gradient-Echo acquired in a sagittal orientation as 1.2 mm thick sections (FOV=25 cm, NEX=1, matrix=256×128, TR=35 ms, TE=9 ms and FA=60°). Scan signal intensities were normalized with reference to white matter intensity. The baseline and follow-up images were co-registered and reformatted in one sinc interpolation step to a 1.5 mm slice thickness oriented to a standard plane.

A three-dimensional region was constructed to span the baseline and the follow-up ventricular CSF that excluded subarachnoid CSF. Following the technique of Fox and Freeborough (1997, J. Mag. Reson. Imag. 7:1069-1075), an automated protocol was applied to the region to segment CSF from surrounding brain tissue at baseline and follow-up. The MRI signal intensities were converted to the CSF volume using the partial volume decomposition method.

After fasting overnight, 30 cc of venous blood and 15 cc of clear CSF were collected using fluoroscopy to guide a 22 gauge beveled LP needle. Samples were centrifuged for 10 min at 1500 rpm at 4° C., aliquoted to 0.25 cc polypropylene tubes, and stored at −80° C. All assays were done blind to clinical data and were batch processed.

A sandwich enzyme-linked immunosorbent assay (ELISA) was used to detect tau phosphorlyated at threonine 231 (p-tau₂₃₁) in CSF. In this assay, tau was captured with two backbone-directed antibodies, tau-1 and CP-27. The captured tau was then detected by CP9, which is specific for p-tau₂₃₁ (Kohnken et al., 2000, Neurosci. Lett. 287:187-190). The detection limit for this assay was 9 pg/ml. The coefficients of variation ranged from 6.0-10.3% (intra-assay) and 11.6-14.4% (inter-assay).

CSF Aβ levels were measured using monoclonal antibody 6E10 (specific to an epitope present on Aβ-16) and to rabbit antisera to Aβ40 and Aβ42, respectively in a double antibody sandwich ELISA (Kohnken et al., 2000, Neurosci. Lett. 287:187-190). The detection limit for Aβ40 and Aβ42 was 10 pg/ml. The reproducibility ranged from 8 to 14% (intra-assay) and 10-18% (interassay).

The load of p-tau₂₃₁ (ng) in the CSF was estimated by: multiplying the p-tau₂₃₁ concentration (pg/ml) by the ventricular volume (ml) and dividing by 1000.

Because of high between subject variability for the CSF measures, the Mann-Whitney U-test was used to test for cross-sectional differences, and longitudinal group interactions. Significant longitudinal effects were followed up using the Wilcoxon Signed Ranks test to examine within group change. For the other measures, the t-test for independent means was used to examine cross sectional data. Longitudinal effects were evaluated using average group differences between follow-up and baseline measures (deltas). Step-wise linear regression models predicting diagnostic group were used to evaluate the unique contribution of the longitudinal dilution correction.

There were no age, education, gender, APOE genotype, or time to follow-up differences found between the normal and MCI groups (see Table 2).

TABLE 2
Demographic Variables

| | Groups | |
|---|---|---|
| Measures | Normal | MCI |
| Number of subjects | 10 | 8 |
| Age (years) Baseline | 62.5 ± 9.2 | 69.8 ± 9.2 |
| Male/female | 5/5 | 3/5 |
| ApoEx4/xx | 2/8 | 3/5 |
| Education (years) | 16.8 ± 1.7 | 14.3 ± 3.2 |
| Follow-up (years) | 1.3 ± 0.4 | 1.3 ± 0.2 |
| GDS Baseline** | 1.5 ± 0.5 | 3.1 ± 0.4 |
| GDS Follow-up** | 1.6 ± 0.5 | 3.2 ± 0.5 |
| MMSE Baseline | 29.4 ± 0.7 | 28.5 ± 1.2 |
| MMSE Follow-up* | 29.9 ± 0.3 | 27.1 ± 3.01 |

*P ≦ 0.05,
**P ≦ 0.01

At both the baseline and the follow-up, both the immediate and delayed paragraph recall were reduced in MCI (P<0.05). No longitudinal change was observed for any neuropsychological measure (P>0.05). One MCI patient converted to very mild AD (GDS=4, MMSE=30).

The ventricular volume was approximately 40% greater in MCI at both baseline (t(16)=−2.1, P<0.05) and at follow-up (t(16)=−2.2, P<0.05). No longitudinal ventricular changes were observed (see Table 3).

TABLE 3

| | Baseline | | | Follow-up | | |
|---|---|---|---|---|---|---|
| Measure | NL | MCI | % Diff | NL | MCI | % Diff |
| VV cc | 29.7 ± 9.8 | 42.3 ± 15.2 | 42* | 32.6 ± 10.3 | 45.7 ± 14.6 | 40** |
| Ptau231 pg/ml | 160.0 ± 190.4 | 534.7 ± 451.8 | 234* | 111.4 ± 158.0 | 563.9 ± 478.0 | 406** |
| Ptau231* VV ng | 5.6 ± 9.1 | 19.7 ± 15.3 | 252** | 4.7 ± 8.6 | 22.8 ± 17.5 | 385**⁺ |
| Aβ 40 pg/ml | 9596 ± 2317 | 12393 ± 2389 | 29* | 8825 ± 2145 | 11876 ± 2909 | 35* |
| Aβ 40* VV ng | 293.8 ± 128.8 | 516.1 ± 187.5 | 76* | 285.3 ± 112.0 | 543.5 ± 241.5 | 91* |
| Aβ 42 pg/ml | 1015.1 ± 448.3 | 943.7 ± 486.7 | −7 | 1040.3 ± 413.2 | 903.7 ± 484.1 | −13 |
| Aβ* VV ng | 29.9 ± 13.6 | 41.8 ± 30.3 | 40 | 31.8 ± 11.9 | 42.9 ± 31.1 | 35 |

VV, ventricle volume; Ptau, p-tau₂₃₁; Aβ amyloid beta; cc, cubic centimeter
% Diff. = cross-sectional difference in MCI relative to NL, Cross-sectional effects
*P ≦ 0.05,
**P ≦ 0.01.
Longitudinal effects:
⁺= P ≦ 0.05

p-tau₂₃₁ levels were elevated in the MCI group at baseline (U=14.0, P<0.05, n=18) and at follow-up (U=6.0, P<0.01, n=18, see Table 3). Aβ40 levels were also significantly elevated in the MCI group at baseline (U=13.0, P<0.05, n=18) and follow-up (U=17.0, P<0.05, n=18). AP42 levels did not differ between the groups. No longitudinal changes were observed for any CSF level measure (P>0.05).

p-tau₂₃₁ loads were elevated in the MCI group at baseline (U=11, P<0.01, n=18) and at follow-up (U=6, P=0.001, n=18). The Aβ40, but not Aβ42, load was significantly elevated in the MCI group at baseline (U=15, P<0.05, n=18) and follow-up (U=9, P<0.01, n=18). In the longitudinal design, a significant group by time interaction was observed only for the p-tau₂₃₁ load (U=14.0, P<0.05, n=18). Follow-up examination showed a significant p-tau₂₃₁ load increase in the MCI group (Z=−2:1, P<0.05, n=8). No longitudinal load effects were observed for the control group.

The longitudinal p-tau load and p-tau level changes were directly compared using two hierarchical linear regression models to predict diagnostic group (reversed orders of entry). Comparing the first entry steps, only the delta p-tau load was related to group membership (R²=0.37, (F[1, 16]=9.5, P<0.01). Comparing the second steps, the delta p-tau load uniquely increased the variance explained by the delta p-tau level (R² change=0.28, F[1, 15]=7.3, P<0.05).

The cross-sectional data show that two markers of AD pathology (p-tau₂₃₁ and Aβ40) are elevated in MCI. Longitudinally, only the p-tau₂₃₁ increases in MCI, as detected by correcting for the change in ventricle size.

### Example 4

### p-tau₂₃₁ in the CSF of Patient's who have AD

Twenty-two patients with the clinical diagnosis of probable AD according to the NINCDS-ADRDA criteria were selected. For comparison of baseline MRI measures, 21 healthy volunteers were selected. Subjects were recruited from the Department of Psychiatry, Alzheimer Memorial Center and Geriatric Psychiatry Branch, Dementia and Imaging Research Section, Ludwig-Maximilian University Munich, Germany. Subject characteristics are given in Table 4.

TABLE 4
Subjects' characteristics

| | Age in years | | Age at onset | MMSE |
|---|---|---|---|---|
| | mean | | mean [SD]/ | mean |
| Group | [SD]^{a}/range | f/m^{b} | range | [SD]^{c}/range |
| Healthy | 60.8 [±8.5] | 11/10 | | 29.4 [±0.7] |
| controls | 50-79 | | | 28-30 |
| (21) | | | | |
| AD patients | 67.8 [±7.9] | 13/9 | 65 [±7] | 23.1 [±4.0] |
| (22) | 53-78 | | 52-74 | 14-29 |

^{a}different between groups, t = 2.8 with 41 df, p < 0.01
^{b}not different between groups, χ² = 0.20 with 1 df, p = 0.66
^{c}different between groups, Mann-Whitney U = 6.5, p < 0.001;

Cognitive impairment in the AD patients was assessed using the Mini Mental State Examination (MMSE). Three patients had moderate (10<=MMSE<20) and 19 patients mild (MMSE >=20) dementia. Twenty AD patients were studied twice with MRI, 2 AD patients had three MRI scans. In the AD patients, length of observation time ranged between 11.3 and 41.0 months (mean 18.4 months; SD 9.4). The average interscan interval was 17.8 months (SD 9.2), ranging between 11.0 and 41.0 months.

Significant medical co-morbidity in the AD patients and controls was excluded by history, physical and neurological examination, psychiatric evaluation, chest X-ray, ECG, EEG, brain MRI, and laboratory tests (complete blood count, sedimentation rate, electrolytes, glucose, blood urea nitrogen, creatinine, liver-associated enzymes, cholesterol, HDL, triglycerides, antinuclear antibodies, rheumatoid factor, VDRL, HIV, serum B12, folate, thyroid function tests and urine analysis). Two AD patients had mild hypertension, no subject had diabetes. With exception of one patient, all AD patients received state of the art anti-dementive treatment during clinical follow up. Of the 21 treated patients, 15 were treated with an acetylcholine-esterase inhibitor, the remaining 6 patients were treated with other drugs, including Akatinol, Ginkgo biloba or other symptomatic nootropic substances. All subjects or the holders of their Durable Power of Attorney signed consent forms to undergo MRI, lumbar puncture and neuropsychological assessment for clinical investigation and research. The protocol was approved by the Ethical Review Board of the Medical Faculty of the Ludwig-Maximilian University, Munich, Germany.

MRI examinations were performed on a 1.5 T Siemens Magnetom Vision MRI scanner (Siemens, Erlangen, Germany). All subjects were investigated with a volumetric T1 weighted sagittal oriented MRI sequence (TR=11.6 ms, TE=4.9 ms, resolution=0.94 by 0.94 by 1.2 mm).

Image preprocessing and segmentation was performed at the McConnell Brain Imaging Center of the Montreal Neurological Institute, Montreal, Quebec, Canada. All images were transferred to a Silicon Graphics workstation (Silicon Graphics, Mountain View, Calif., USA). Prior to volumetric measurements, all MRI volumes were corrected for image intensity non-uniformities, mapped by linear stereotaxic transformation into coordinates based on Talairach atlas, and resampled onto a 1 mm voxel grid. The correction for image intensity has been proven to recover most of the artifacts present in MR images. This preprocessing increases inter- and intra-rater reliability of volumetric measurements and corrects for effects of whole brain atrophy. This is of particular importance when analyzing longitudinal datasets, because reduced measurement reliability at follow up due to greater overall atrophy would lead to systematic non-biological intra-individual effects.

Volumetric analysis was performed with the interactive software package DISPLAY developed at the Brain Imaging Center. This program allows simultaneous visualization and segmentation of volumes in coronal, sagittal and horizontal orientations.

The anatomical boundaries used for HC segmentation have been described in detail before. FIG. 2 shows the localization and morphology of the hippocampus. In short, the following procedures were employed.

The most posterior part of the HC was defined as the first appearance of gray matter inferiomedial to the trigone of the lateral ventricle (TLV) in the caudorostral extent of the HC.

The lateral border in the tail of the HC was the TLV. Medially, the border of the HC was identified by white matter. Moving further anterior, an arbitrary border was defined for the superior and medial border of the HC, in order to differentiate HC gray matter from the gray matter of the Andreas Retzius gyrus, the fasciolar gyrus, and the crus of the formix. The inferior border of the HC at this point was again identified by white matter.

In both the HC tail and body, the white matter band at the superolateral level of the HC, the fimbria, was included. The dentate gyrus, located in between the four CA regions in the hippocampal formation, together with the CA regions themselves and the subiculum, were included. The lateral border at this point was identified by the inferior horn of the lateral ventricle. The quadrogeminal cistern defined the superomedial border of the HC.

The most important structures for identification of lateral, anterior and superior borders of the HC head were the uncal recess of the inferior horn of the lateral ventricle and the alveus. Besides the coronal view, the sagittal and horizontal views were employed for identification of the anterior border of the HC head.

Inter-rater reliability was assessed using four raters independently measuring the same set of 5 MRI scans. The coefficient of variation between the raters ranged between 1.8% and 2.2% for right and left hippocampus. The intraclass correlation coefficients ranged between 0.94 and 0.86 for right and left hippocampus. The intra-rater reliability was determined by one rater measuring four times the same set 5 MRI scans within a previously not measured set of MRI scans. The coefficients of variation within rater ranged between 2.2% and 2.4% for right and left hippocampus. The intraclass correlation coefficients ranged between 0.91 and 0.94 for right and left hippocampus.

CSF samples were taken by lumbar puncture and processed immediately. Aliquots were stored at −80° C. until further examination. The detailed CSF protocol has been described previously.

Tau protein levels were measured using enzyme-linked immunosorbent assays (ELISA) as described above.

Differences in hippocampus and amygdala volumes were compared between groups (AD and controls) using Student's t-test. To control for potential effects of age, between-group differences in hippocampus volumes were assessed using multiple regression analysis with volumes as dependent and age and diagnosis as predictor variables.

Rates of atrophy of left and right hippocampus were determined with mixed effect regression models using SAS 8.02 Proc Mixed software (SAS Institute Inc., Cary, N.C., USA). In a first step, each volume (right and left hippocampus) was predicted by a mixed effect regression term, incorporating random-effect terms to account for subject and subject by time differences in volumes (random trend model). The random trend model conceptualizes each patient's volume change as following a straight line parameterized by a slope and an intercept. The slope measures the patient's rate of atrophy, the intercept the patient's baseline volume. Individual slopes and intercepts are regarded as a random sample of a normal population with unknown mean and variance. Individual slopes were determined by iterative restricted maximum likelihood estimation, taking into account the information of the entire sample to determine individual rates of change. From these models, we derived individual random regression coefficients of time related change in hippocampus volumes. In a second step, additional terms were included into the random effect model to account for effects of p-tau₂₃₁ levels on baseline volume and on individual rates of change in volume. An additional random trend model was used to determine the individual rates of decline in MMSE score

Spearman's rank correlation and Pearson's product moment correlation were used for correlation analyses.

Hippocampus volumes were significantly reduced in AD patients compared to controls (p<0.003 for all comparisons).

These effects remained significant after controlling for age differences between groups using a multiple regression model (p<0.05). Hippocampal volumes are shown in Table 0.5. In the AD group, levels of p-tau₂₃₁ were 729.6 (SD 404.3) pg/ml, and levels of t-tau were 608.1 (SD 314.6) pg/ml. Annual rates of atrophy in the AD patients were about 14% for right and left hippocampus (Table 6).

TABLE 5
Hippocampus volumes

| | healthy | | % difference |
|---|---|---|---|
| | controls | AD patients | AD vs. |
| Region | (n = 21) | (n = 22) | controls |
| left | 3158.5 [SD | 2267.2 [SD | −28%* |
| hippocampus | 582.4] | 640] | |
| right | 3328.0 [SD | 2262.7 [SD | −32%* |
| hippocampus | 556.4] | 651] | |

Mean [±SD] volumes of left and right amygdala and hippocampus in mm³.
*significant difference between AD patients and controls after controlling for age (p < 0.001)

TABLE 6
Rates of atrophy of hippocampal volumes in AD patients (n = 22)

| | annual %-rates of |
|---|---|
| region | atrophy |
| left hippocampus | −13.8 [SD 5.2]* |
| right hippocampus | −14.3 [SD 5.7]* |

Mean [±SD] annual percent rates of atrophy for hippocampal volumes based on random trend model estimates
*significantly different from zero slope, p < 0.001

There was a significant effect of p-tau₂₃₁ and t-tau on baseline volumes of left hippocampus (p-tau₂₃₁: beta=0.65, p<0.002; t-tau: beta=0.50, p<0.01) and right hippocampus (p-tau₂₃₁: beta=0.63, p<0.001; t-tau: beta=0.46, p<0.03), with higher tau protein levels corresponding to higher baseline hippocampus volumes (FIG. 3). Increased levels of p-tau₂₃₁ were significantly correlated with higher rates of atrophy of left hippocampus (beta=−0.36, p<0.001) and right hippocampus (beta=−0.31, p<0.02) (FIG. 4). T-tau levels were not correlated with rates of atrophy. Average rate of decline of MMSE score was −1.32 (SD 1.05) points per year in AD. Higher rates of MMSE score decline correlated significantly with higher rates of atrophy of left and right hippocampus volumes (rho=0.52 and 0.47, p<0.05). There was no effect of levels of tau proteins on baseline MMSE scores or on rates of decline in MMSE scores in the AD patients.

There was no effect of age on rate of atrophy in AD or on baseline volumes in AD and healthy controls. There was no effect of baseline MMSE score, age at onset and disease duration on baseline volumes or rates of volume loss in AD patients. Treatment (coded as “no treatment”, “acetylcholine-esterase inhibitors”, “other drugs”) had no effect on rates of MMSE score decline or atrophy in AD. There was no effect of age, age at onset, disease duration or MMSE score at baseline on CSF levels of t-tau and p-tau₂₃₁ in AD.

Patient and control groups were matched for gender distribution, but were different in age (Table 4).

The data demonstrate a significant effect of baseline levels of tau proteins on baseline volumes of hippocampus, and of p-tau₂₃₁ on progression of hippocampus atrophy over time. These findings agree with the hypothesis that tau protein levels in CSF may reflect the degree of axonal and neuronal destruction in AD, which is the main determinant of subsequent morphological disease progression as visualized with structural MRI.

Levels of p-tau₂₃₁ predicted the rate of subsequent hippocampus atrophy, accounting for about 20% of variability in individual rates of atrophy. Levels of t-tau were not predictive for subsequent rates of atrophy. The degree of neuronal degeneration at a given time period may be highly predictive for the rate of neuronal loss in the subsequent time period. The rate of neuronal loss in AD, however, can be assessed in vivo by MRI based measurement of atrophy, since it has been shown that hippocampus atrophy in MRI accounts for 80% of variability of neuronal cells numbers in hippocampus in AD. On this basis, the significant correlations between p-tau₂₃₁ levels and rates of hippocampal atrophy suggest that CSF levels of p-tau₂₃₁ can serve as a state marker for the degree of neuronal destruction in AD which in turn determines subsequent rates of neuronal loss and regional atrophy.

It should be understood that the foregoing disclosure emphasizes certain specific embodiments of the invention and that all modifications or alternatives equivalent thereto are within the spirit and scope of the invention as set forth in the appended claims.

1-3. (canceled)

4. A method of diagnosing a patient who is likely to develop Alzheimer's Disease (AD) comprising measuring the level of p-tau in the CSF of a patient, wherein a CSF p-tau level of at least about 215 pg/ml in the patient is indicative of a patient who is likely to convert to AD.

5. The method of claim 4, wherein the patient has mild cognitive impairment (MCI).

6. The method of claim 4, wherein the level of p-tau is least about 250 pg/ml, at least about 300 pg/ml, or at least about 350 pg/ml.

7. The method of claim 4, wherein the p-tau is tau protein phosphorylated at threonine 231 (p-tau₂₃₁).

8. A method of diagnosing a patient as having mild cognitive impairment (MCI) comprising:
a) determining the level of phosphorylated tau protein in a sample of cerebrospinal fluid (CSF) taken from the patient;
b) determining the CSF level of p-tau obtained from at least one control patient;
c) comparing the CSF level of p-tau from the patient with the CSF level of p-tau obtained from the at least one control patient, wherein the patient is diagnosed as having MCI if the CSF level of p-tau is greater in the patient than in the at least one control patient.

9. The method of claim 8, wherein the p-tau is tau protein phosphorylated at threonine 231 (p-tau₂₃₁).

10. A method of predicting the rate of neuronal degeneration in a patient comprising determining the level of phosphorylated tau protein in a sample of cerebrospinal fluid (CSF) taken from the patient, wherein the patient has Alzheimer's Disease.

11. The method of claim 10, wherein the p-tau is tau protein phosphorylated at threonine 231 (p-tau₂₃₁).

12-16. (canceled)

17. A method of predicting cognitive decline in a patient, comprising the steps of:
a) obtaining a biological sample from a patient;
b) determining the level of phosphorylated tau protein (p-tau) in the biological sample;
c) identifying the patient as being likely to cognitively decline if the level of p-tau in the biological sample is about 143 pg/ml or higher.

18. The method of claim 17, wherein the patient has mild cognitive impairment (MCI).

19. The method of claim 17, wherein the p-tau is tau protein phosphorylated at amino acid 175, 181, 185, 199, 202, 214, 231, 235, 262, 396, 404, 409, or 422.

20. The method of claim 19, wherein the p-tau is tau protein phosphorylated at threonine 231 (p-tau₂₃₁).

21. The method of claim 17, wherein the biological sample is cerebrospinal fluid (CSF).

22-29. (canceled)

30. A method of measuring the effectiveness of a pharmaceutical composition as an agent for treating a patient having a condition associated with dementia, comprising the steps of:
a) determining the level of p-tau protein in a biological sample obtained from the a patient;
b) administering an amount of a pharmaceutical composition to the patient;
c) repeating step (a) using a subsequently-collected biological sample obtained from the patient;
d) comparing the level of p-tau protein determined in step (a) with the level of p-tau protein determined in step (c), wherein the effectiveness of the pharmaceutical composition is monitored by detecting no change in the level or p-tau or a decrease in the level of p-tau in the subsequently-collected biological sample compared with the biological sample from step (a).

31. The method of claim 30, wherein the condition is Alzheimer's Disease or Mild Cognitive Impairment.

32. The method of claim 30, wherein the p-tau is tau protein phosphorylated at amino acid 175, 181, 185, 199, 202, 214, 231, 235, 262, 396, 404, 409, or 422.

33. The method of claim 32, wherein the p-tau is tau protein phosphorylated at threonine 231 (p-tau₂₃₁).

34. The method of claim 30, wherein the biological sample is cerebrospinal fluid (CSF).

35-45. (canceled)

46. The method of claim 17, further comprising treating the patient identified in step (c) with an anti-dementia drug.

47. (canceled)

48. A method of treating a patient for cognitive decline comprising diagnosis according to the method of claim 17 and administering an anti-dementia drug to the patient.

49. (canceled)
